(19) United States
(12) Patent Application Publication
(10) Pub. No.: US 2026/0174510 A1
(43) Pub. Date: Jun. 25, 2026

(54) CART FOR TRACKING A CATHERIZATION TABLE

(71) Applicant: Siemens Heatlhineers Endovascular Robotics, Inc., Newton, MA (US)

(72) Inventor: Eric Klem, Lexington, MA (US)

(21) Appl. No.: 18/989,616

(22) Filed: Dec. 20, 2024

(51) Int. Cl. A61B 34/30

(52) U.S. Cl. CPC A61B 34/30

(57) ABSTRACT

A system includes a base, a first carriage coupled to the base and configured to move in a first direction with respect to the base, a second carriage coupled to the first carriage and configured to move along the first carriage in a second direction substantially perpendicular to the first direction, a third carriage coupled to the second carriage and configured to move along the second carriage in a third direction substantially perpendicular to the first direction and to the second direction, and a tilting mechanism comprising a table interface. Movement of a table coupled to the table interface in the first direction causes the first carriage to move in the first direction, movement of the table in the second direction causes the second carriage to move in the second direction, movement of the table in the third direction causes the third carriage to move in the third direction, and tilting of the table causes the tilting mechanism and the robotic drive to tilt.

### BACKGROUND

Vascular disease may be treated in a variety of ways. For example, cardiovascular disease may be treated with bypass surgery. In contrast to surgical treatments, catheter-based interventional procedures such as angioplasty present a potentially-safer and less-invasive alternative.

Robotic catheter systems perform catheter-based interventional procedures via motor-driven manipulation of catheters, guidewires and other elongated medical devices (EMDs). During a procedure, drive elements of a robotic drive are operated to impart desired movement to EMDs which are mounted therein. The movement may consist of rotation, linear translation, and/or any other type of movement.

An articulated arm typically holds a robotic drive adjacent to a patient access site during a catheter-based interventional procedure. Conventionally, the arm remains attached to a rail mounted to the patient table during the procedure. The total weight of the arm and drive must therefore be managed in order to avoid overloading the rail and table. Between procedures, the arm and drive are removed from the rail and transferred to a floor or another storage area. Removal and transfer of the arm and drive can be difficult and cumbersome.

For convenience, it may be desirable to mount the arm to a structure other than the table, such as a floor, a ceiling, or a movable cart. Since the table (and a patient positioned thereon) may move during a procedure, mounting the arm to a structure other than the table necessitates visually tracking the patient access site in three-dimensional space and movement of the arm and robotic drive in correspondence with the tracking. Mechanisms for executing the tracking and the corresponding movement of the arm and robotic drive add significantly to the complexity of the system and increase the chance of errors.

Systems are desired which efficiently and accurately react to patient and table movement during a robotic catheter-based interventional procedure while providing the conveniences of mounting a robotic drive to a structure other than the table. Such systems preferably address movements including table pitch and deflections.

### BRIEF DESCRIPTION OF THE DRAWINGS

Embodiments will become more fully understood from the following detailed description, taken in conjunction with the accompanying drawings, wherein the reference numerals refer to like parts in which:

FIG. 1 is a perspective view of a robotic drive, arm and cart in accordance with some embodiments;

FIGS. 2 and 3 are views showing different vertical positions of a vertical carriage in accordance with some embodiments;

FIGS. 4 and 5 are views showing different longitudinal positions of a longitudinal carriage in accordance with some embodiments;

FIGS. 6 and 7 are views showing different transverse positions of a transverse carriage in accordance with some embodiments;

FIG. 8 is a view illustrating tilting of a transverse carriage and of a robotic drive and arm mounted thereon in accordance with some embodiments;

FIG. 9 illustrates a table interface of a transverse carriage in accordance with some embodiments;

FIG. 10 is a detailed view of a table interface of a transverse carriage in accordance with some embodiments;

FIG. 11 is a perspective view of a table in a procedure theatre;

FIGS. 12 and 13 are perspective views of a robotic drive, arm, cart and table in accordance with some embodiments;

FIG. 14 is a perspective view of a cart coupled to a table in an initial position in accordance with some embodiments;

FIG. 15 is a perspective view of a cart coupled to a table moved transversely from an initial position in accordance with some embodiments;

FIGS. 16 and 17 are perspective views of a cart coupled to a table moved longitudinally from an initial position in accordance with some embodiments;

FIG. 18 is an elevation view of a cart coupled to a table in an initial position in accordance with some embodiments;

FIGS. 19 and 20 are elevation views of a cart coupled to a table tilted from an initial position in accordance with some embodiments;

FIGS. 21A and 21B depict a mechanism for coupling a carriage to a table in accordance with some embodiments;

FIGS. 21C and 21D are cross-sectional views depicting the coupling of a carriage to a table in accordance with some embodiments;

FIG. 22 is a perspective view of a locking arm in accordance with some embodiments;

FIGS. 22A and 22B illustrate mechanisms for facilitating and preventing longitudinal movement of a carriage in accordance with some embodiments;

FIGS. 23A and 23B illustrate locking longitudinal movement of a carriage in accordance with some embodiments;

FIGS. 24 and 25 illustrate mechanisms for providing rotation and transverse movement of a carriage in accordance with some embodiments; and

FIGS. 26, 27A and 27B illustrate locking the movement of a carriage in accordance with some embodiments.

### DETAILED DESCRIPTION

The present inventor has recognized the need to address changes in table pitch in a manner which does require visual tracking of the patient rail. Visually tracking the position of the rail is not equivalent to visually tracking the position of the access site, particularly in the case of a procedure involving large pitch angle variations and/or longitudinal table movement. Due to the length of a typical patient table, a slight change to the pitch of the table results in significant vertical movement of the access site. Moreover, longitudinal table movement may cause deflections which also cause vertical movement of the access site. In particular, movement of a long and heavy table surface relative to a narrow table pedestal changes the moment arm and therefore the bending moment, which in turn causes deflections of the pedestal and corresponding vertical movements of the access site which vary with longitudinal table position.

Some embodiments address the foregoing by providing a system including a movable base, a first carriage coupled to the base and configured to move in a first direction with respect to the base, a second carriage coupled to the first carriage and configured to move along the first carriage in a second direction substantially perpendicular to the first direction, and a third carriage coupled to the second carriage and configured to move along the second carriage in a third direction substantially perpendicular to the first direction and to the second direction. The third carriage includes a mount for a robotic drive and a table interface for coupling the third carriage to a patient table.

Advantageously, movement of a table coupled to the interface in the first direction causes the first carriage to move in the first direction, movement of the table in the second direction causes the second carriage to move in the second direction, movement of the table in the third direction causes the third carriage to move in the third direction, and tilting of the table causes the third carriage and a robotic drive mounted to the third carriage to tilt.

By virtue of the foregoing, the position of the robotic drive with respect to a patient positioned on the table (and, in particular, with respect to a catheter insertion point on the patient) may be maintained during a procedure. The position is maintained if the table is moved longitudinally, moved transversely, or tilted in response to an operator instruction, or if the table moves inadvertently, e.g., due to table deflection.

Some embodiments advantageously allow storage and transport of the robotic drive, as well as coupling of the third carriage to a table, without detaching the robotic drive from the third carriage. Embodiments may also eliminate the need for electronic systems to detect the position of the table and mechanical systems to move the robotic drive in response to the detected position.

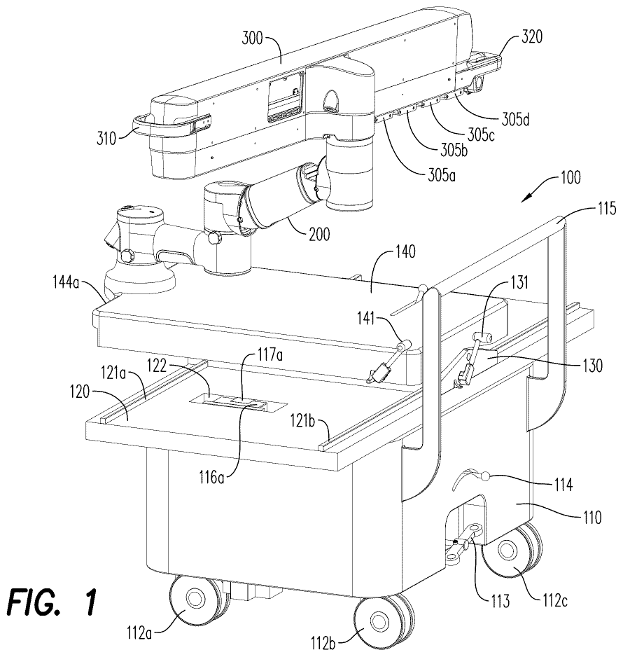

FIG. 1 is a perspective view of movable cart 100, arm 200 and robotic drive 300 in accordance with some embodiments. The components of FIG. 1 may be used to perform catheter-based interventional procedures, e.g., percutaneous intervention procedures such as percutaneous coronary interventions (e.g., to treat STEMI), neurovascular interventions (e.g., to treat an aneurysm), and peripheral vascular interventions (e.g., for critical limb ischemia (CLI), etc.). Catheter-based procedures may include diagnostic catheterization procedures during which one or more catheters or other EMDs are used to aid in the diagnosis of a patient's disease. An example robotic drive is described in application number PCT/US2021/070042, which is hereby incorporated by reference in its entirely. In particular, in some embodiments, the robotic drive may comprise a plurality of drive modules moveable along an axis of the drive module, for example, a longitudinal axis extending substantially along a patient table. The drive modules may each comprise at least one cassette, which may be disposable. The cassette and drive modules may be configured to drive at least one elongated medical device (EMD), which may be catheters, guidewires, microwires and catheters, etc. The drive modules and cassettes may be disposed or mounted in a “vertical” orientation such that the drive module to which a cassette is mounted is located off to the side and no longer positioned between a cassette and the patient. A vertical orientation may mean that a device module includes a cassette that is mounted to a drive module such that a front face or side of the cassette is parallel to a front face or side (i.e., a mounting surface) of the drive module. The vertical mounting orientation of the cassette eliminates the need for the drive module to be placed under the device axis and between the elongated medical device and the patient. Rather, only a portion of the cassette is positioned between the elongated medical device and the patient. The device module may be connected to a stage that is moveably mounted to a rail or linear member. The drive module may include a coupler that is used to provide a power interface to the cassette to, for example, rotate an elongated medical device (not shown) positioned in the cassette In some procedures, a contrast media is injected into one or more arteries through a catheter and an image of the patient's vasculature is acquired while the contrast media resides therein.

As used herein, the term EMD refers to, but is not limited to, catheters (e.g., guide catheters, microcatheters, balloon/stent catheters), wire-based devices (e.g., guidewires, microwires, proximal pushers for embolization coils, stent retrievers, self-expanding stents, flow divertors, etc.), and medical devices comprising any combination of these.

Generally, robotic drive 300 may be loaded with EMDs which are appropriate for a given procedure. Embodiments are not limited to robotic drive 300 of FIG. 1. A robotic drive for use in conjunction with some embodiments is capable of imparting movement to one or more EMDs in response to instructions comprising electronic signals, as opposed to manual manipulation of the one or more EMDs by a human operator.

The electronic signals may be generated in response to operator manipulation of an input panel located on the robotic drive and/or controls of a control station such as an operator cockpit or a handheld device. A control station may be located proximate to the robotic drive (e.g., near a patient undergoing a procedure) and/or away from the robotic drive (e.g., behind shielding to protect the operator from radiation emitted from imaging devices used during a procedure). A control station may also be used to control an imaging device and a patient table during a procedure as is known in the art.

Robotic drive 300 includes multiple drive modules 305a-d. A respective cassette (not shown) may be mounted to each drive module 305a-d during a procedure. Each cassette may include elements to support an EMD loaded therein and move (e.g., rotate and/or translate) the EMD in one or more degrees of freedom. Each drive module 305a-d includes at least one coupler to interface with such elements in each cassette. Each drive module 305a-d also includes a motor (not shown) that is used to rotate its corresponding coupler. Accordingly, rotation of a coupler by a motor of its corresponding drive module 305a-d may cause the coupler to drive mechanisms in the cassette mounted thereto to cause, for example, rotation of an EMD loaded in the cassette. A cassette may provide a sterile interface between at least one EMD and a drive module directly or through a device adapter.

Each drive module 305a-d is movable in a linear direction independently of each other drive module 305a-d. Independent linear movement of drive modules 305a-d results in independent linear movement of any EMDs loaded within cassettes coupled to drive modules 305a-d.

Drive modules 305a-d are configured such that they are in a vertical configuration with respect to a patient during a procedure. A vertical orientation reduces the distance between robotic drive 300 and the patient and the distance between a longitudinal axis of robotic drive 300 and an introducer sheath.

Robotic arm 200 is used to position and support robotic drive 300 before, during and after a procedure. After positioning a patient on a table, the articulated members of robotic arm 200 are manipulated (e.g., manually and/or via electronic signals) to move robotic drive 300 to a position relative to the patient which is appropriate for a given procedure. Once so positioned, the joints of robotic arm 200 can be locked to prevent further movement. An exemplary robotic arm that may be used with the present invention is that shown and described in U.S. Ser. No. 17/812,508 (U.S. Pat. No. 11,906,009), which is hereby incorporated by reference in its entirety.

Referring to FIGS. 1-3, cart 100 includes base 110 to which casters 112a-112d are attached. Handle 115 is attached to base 110 and, along with casters 112a-112d, facilitates moving of cart 100 and any elements attached thereto to desired locations. Base 110 also includes foot pedal 113 for controlling a locking function of casters 112a-112d as is known in the art. For example, depression of one side of pedal 113 may lock casters 112a-112d in their current position (i.e., preventing swiveling and rolling motions) while depression of the other side of pedal 113 may unlock casters 112a-112d so they may swivel and roll freely. A middle (i.e., horizontal) position of pedal 113 may engage a “steering” mode in which casters 112a and 112b are unlocked and casters 112c and 112d are prevented from swiveling but roll freely.

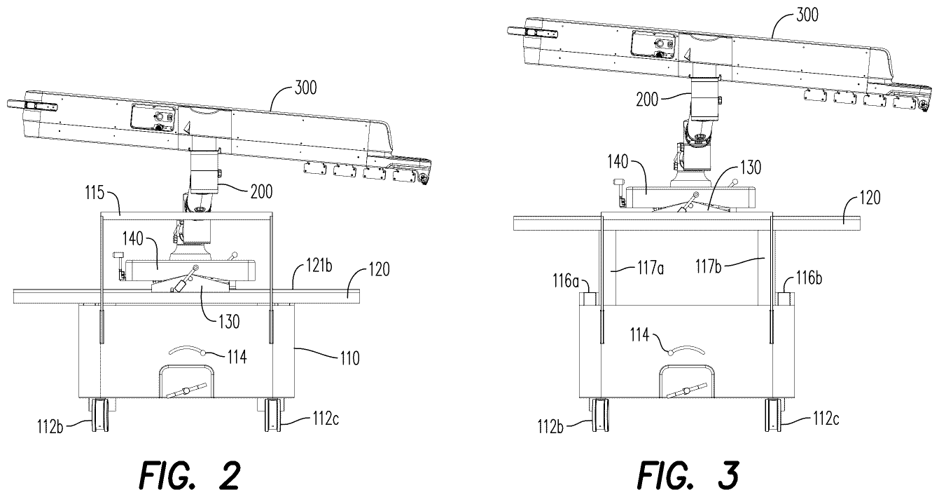

Vertical carriage 120 is coupled to base 110 via supports 116a, 116b, 117a and 117b. Supports 116a and 116b are fixed and extend vertically from base 110, and supports 117a and 117b are nested within respective ones of supports 116a and 116b. While in the position shown in FIGS. 1 and 2, support 116a and an upper portion of support 117a reside in opening 122 of carriage 120.

Supports 117a and 117b may be moved vertically with respect to base 110 as shown in FIG. 3. This movement causes carriage 120 to move vertically with respect to base 110. During an interventional procedure, this vertical movement may be in a direction roughly parallel to the vertical axis of a patient table. Supports 117a and 117b may be telescopic, in which case lower portions of supports 117a and 117b remain fixed with respect to base 110 while upper portions of supports 117a and 117b are extendable upward to move carriage 120 vertically.

Embodiments may comprise any suitable system for moving supports 117a and 117b vertically, including electromechanical systems. According to some embodiments, an operator may manually move supports 117a and 117b by lifting carriage 120. Vertical carriage 120 may include a spring mechanism to counter its own weight and the weight of any elements attached thereon (e.g., carriage 130, carriage 140, arm 200 and drive 300), to assist the manual raising of carriage 120 by an operator and to reduce a force applied to a table coupled to carriage 120.

In one example, vertical carriage 120 is prevented from extending vertically from base 110 when carriage 120 and lever 114 are in the position shown in FIGS. 1 and 2. Lever 114 must be moved to the unlocked position shown in FIG. 3 prior to raising carriage 120. According to some embodiments, vertical movement of carriage 120 may be locked only when carriage 120 is in the fully-lowered position shown in FIGS. 1 and 2.

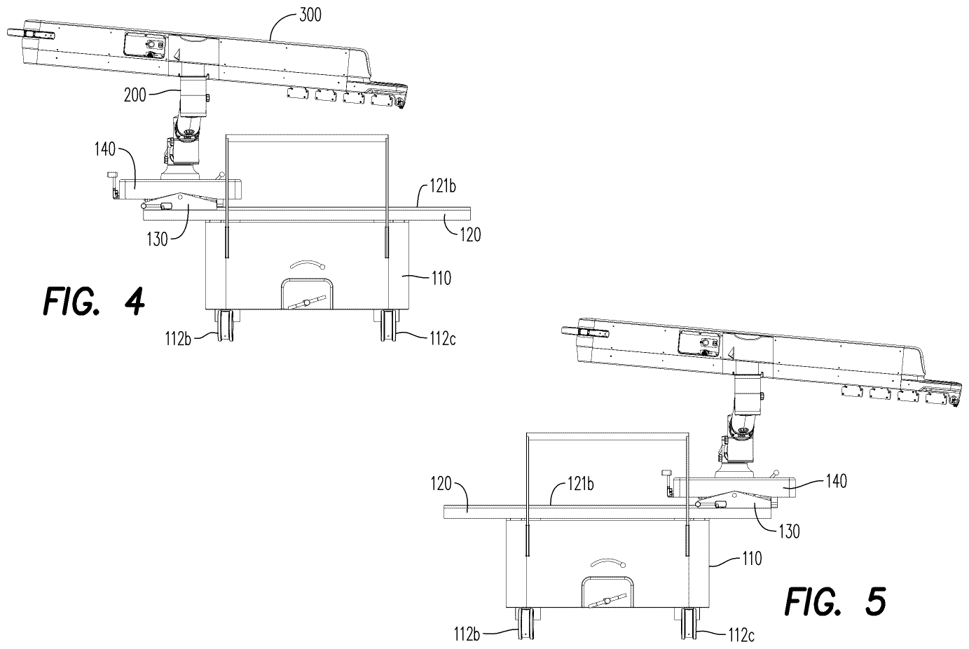

Rails 121a and 121b are attached to carriage 120 and extend longitudinally thereon. Longitudinal carriage 130 is configured to move longitudinally with respect to carriage 120 along rails 121a and 121b as shown in FIGS. 4 and 5. This longitudinal movement also moves any elements disposed on carriage 130 (e.g., carriage 140, arm 200 and drive 300) in the longitudinal direction. During an interventional procedure, the longitudinal direction is a direction roughly parallel to the longitudinal axis of a patient table. An underside of longitudinal carriage 130 (not shown) includes wheels, bearings, and/or other suitable mechanisms for interfacing with rails 121a and 121b and for translating smoothly thereon.

Longitudinal carriage 130 includes locking arm 131 shown in a locked position in FIGS. 1-3. When locking arm 131 is in the locked position, longitudinal carriage 130 is prevented from moving along rails 121a and 121b. When locking arm 131 is in an unlocked position as shown in FIGS. 4 and 5, carriage 130 may be manually moved to any position along rails 121a and 121b. According to some embodiments, locking arm 131 may be engaged in the locked position to lock the longitudinal movement of carriage 130 when carriage 130 is positioned at any position along rails 121a and 121b.

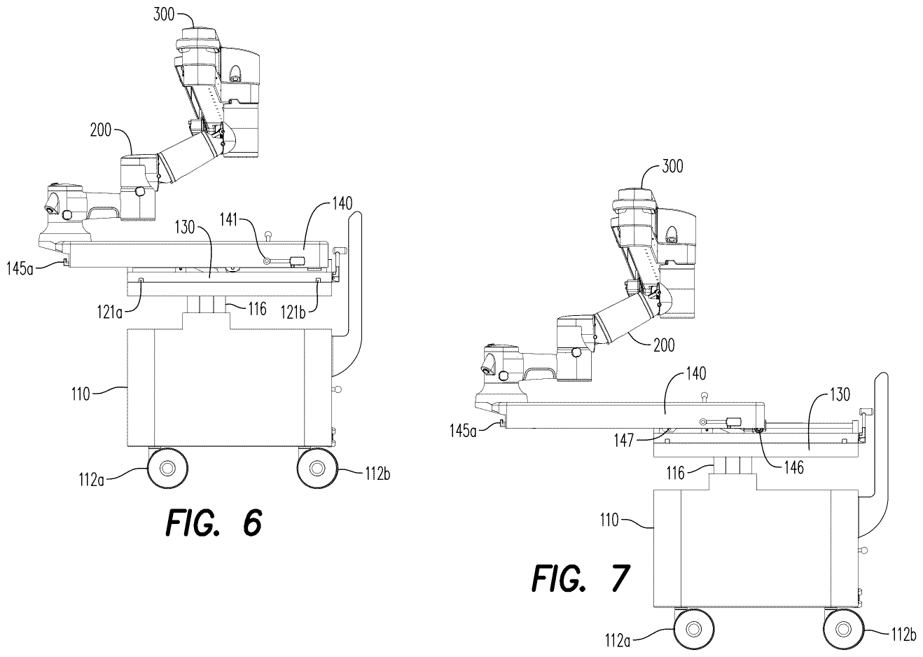

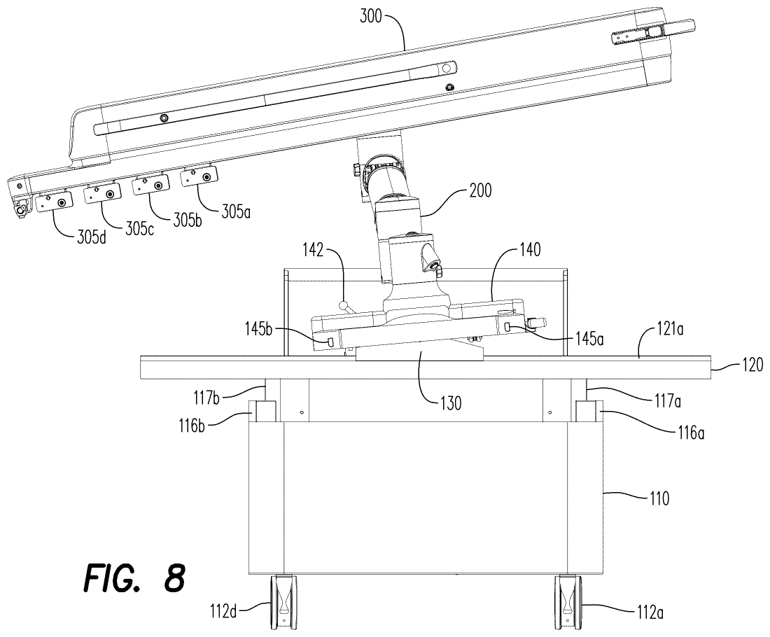

Transverse carriage 140 is coupled to carriage 130 and is configured to move in a transverse direction with respect to carriage 130 as shown in FIGS. 6 and 7. The directions in which carriages 120, 130 and 140 may move are therefore roughly perpendicular to one another. Transverse movement of carriage 140 causes any elements disposed thereon (e.g., arm 200 and drive 300) to also move in the transverse direction. The transverse direction is roughly perpendicular to the longitudinal axis of a patient table during a procedure.

Locking arm 141 of transverse carriage 140 is shown in a locked position in FIGS. 1-5 and in an unlocked position in FIGS. 6 and 7. Placing locking arm 141 in the locked position prevents transverse movement of transverse carriage 140 with respect to longitudinal carriage 130. According to some embodiments, locking arm 141 may be engaged in the locked position to lock the transverse movement of carriage 140 at any position along its range of transverse travel.

According to some embodiments, transverse carriage 140 is rotatable about a longitudinal axis of carriage 130 as shown in FIG. 8. In other words, transverse carriage 140 may tilt from a horizontal position in a clockwise or a counterclockwise direction about an axis parallel to the above-described transverse direction of travel. During a procedure, this tilting may mirror the tilting of a patient table about a transverse axis of the patient table. As a result, any elements which are fixed to carriage 140 (e.g., arm 200 and drive 300) would also tilt in a manner which mirrors the tilting of the patient table.

Locking arm 141 may also lock and unlock the ability of transverse carriage 140 to rotate as described. Carriage 140 may be locked at any rotational position. According to some embodiments, carriage 140 includes a spring mechanism to bias carriage 140 into the horizontal position when unlocked but allows carriage 140 to tilt in response to table movement as described below.

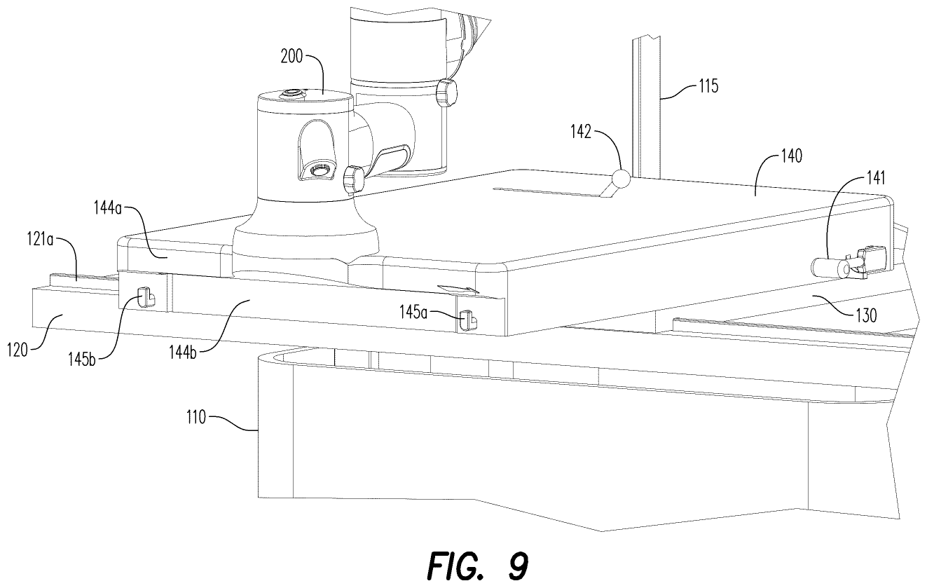

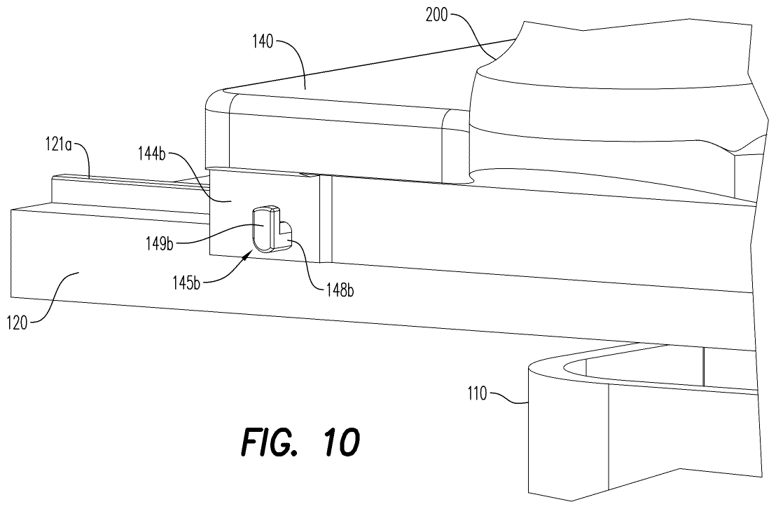

FIGS. 9 and 10 show projections 145a and 145b extending from lower ledge 144b of carriage 140. Projections 145a and 145b and clamp actuator 142 are used to clamp carriage 140 to a patient table during a procedure. Movement of actuator 142 to the position shown in FIG. 9 causes projection 145b to rotate from a horizontal orientation to the depicted vertical orientation.

As shown in FIG. 10, portion 148b of projection 145b extends outward from lower ledge 144b and portion 149b is perpendicular to portion 148b. Each of projections 145a and 145b may be coupled to a spring mechanism (not shown) which biases it toward the horizontal orientation.

When carriage 140 is clamped to a table and carriages 120, 130 and 140 are unlocked, any vertical, longitudinal, transverse movement or pitch of the table causes carriages 120, 130 and 140 to move such that a position of an end of robotic drive 300 with respect to a catheter insertion point remains substantially unchanged.

FIGS. 9 and 10 also illustrate the coupling of one end of arm 200 to upper ledge 144a of carriage 140. The other end of arm 200 is attached to robotic drive 300. Consequently, when the joints of arm 200 are locked in place, movement of ledge 144a of carriage 140 results in corresponding movement of drive 300.

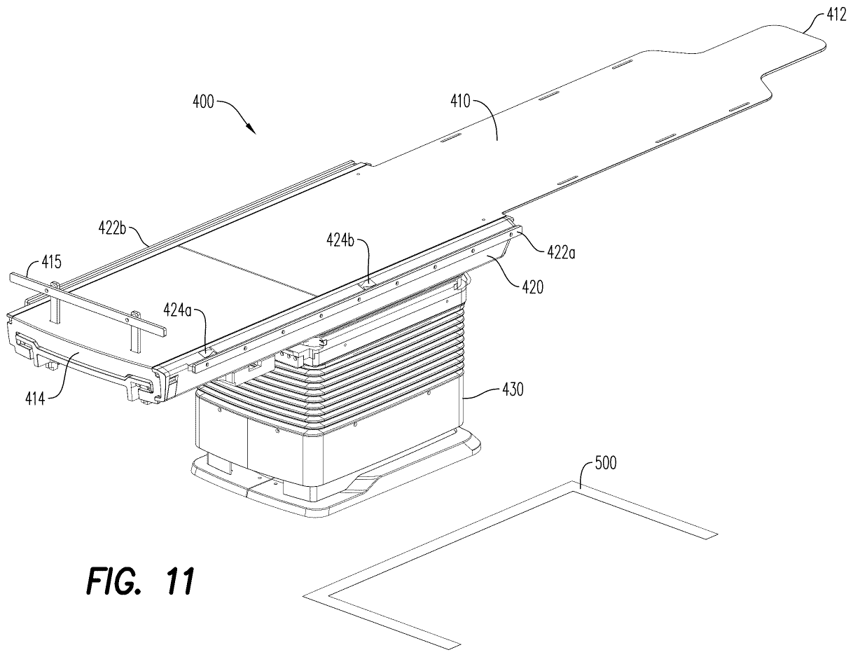

FIG. 11 is a perspective view of table 400 which supports a patient during an interventional procedure. Table 400 consists of support 410, sled 420 and pedestal 430. Sled 420 and pedestal 430 are operable to move support 410 with multiple degrees of freedom, for example, vertical, longitudinal, transversal, roll, pitch, and yaw. For reference, the longitudinal axis of support 410 is defined by ends 412 and 414 of support 410.

Mechanisms within sled 420 may operate to move support 410 in the longitudinal direction while sled 420 remains fixed with respect to pedestal 430. Mechanisms within pedestal 430 may operate to move sled 420 and support 410 vertically and/or in the transverse direction. Pedestal 430 may also be operated to tilt sled 420 and support 410 about a transverse axis.

Rails 422a and 422b are fixed to sled 420. According to some embodiments, carriage 140 of cart 100 may be fixedly coupled to sled 420 during a procedure. More specifically, projections 145a and 145b may be moved adjacent to openings between rail 422a and sled 420 and rotated to move into the openings, resulting in clamping of carriage 140 to sled 420.

Positioning guide 500 is a marking on a floor adjacent to table 400. Guide 500 may comprise tape, paint, a sticker and/or any other suitable marking media. Guide 500 indicates a position in which cart 100 may be disposed in order to couple the cart 100 to table 400. Table 400 may also be moved to a known position so that cart 100 couples to a desired portion of table 400. Additionally or alternatively, rail 422a includes marks which are aligned to projections 145a and 145b by moving table 400 and/or cart 100.

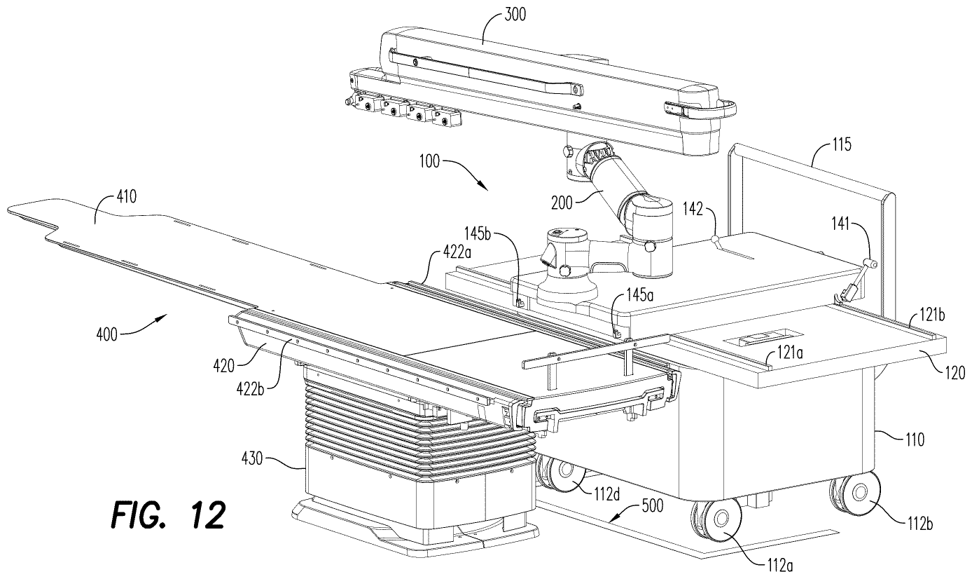

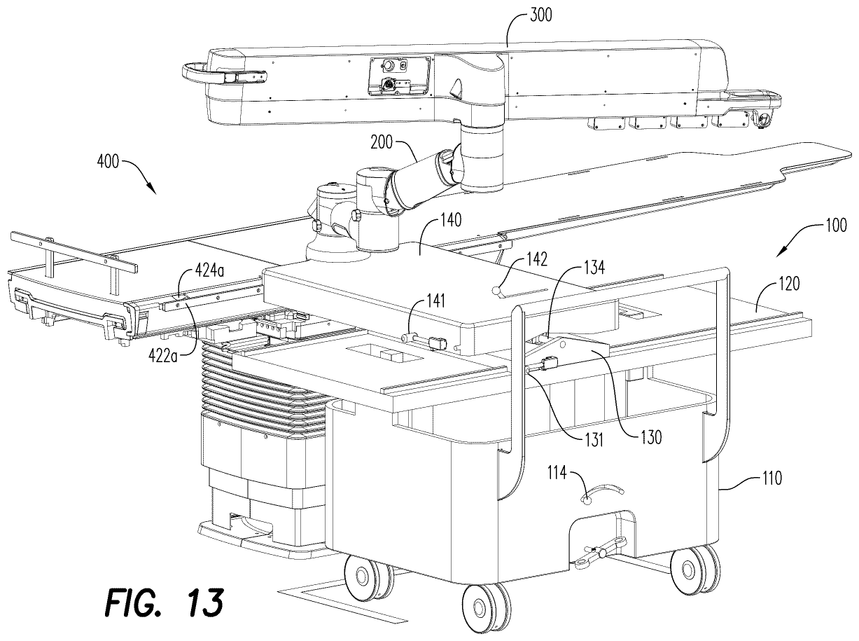

FIGS. 12 and 13 illustrate coupling of cart 100 to table 400 according to some embodiments. As shown in FIG. 12, an operator locks vertical movement of carriage 120 using lever 114, locks longitudinal movement of carriage 130 using locking arm 131, and locks transverse and tilting movement of carriage 140 using locking arm 141. Casters 112a-112d are unlocked using pedal 113 and handle 115 is used to move cart 100 into alignment with positioning guide 500. Casters 112a-112d are then locked using pedal 113.

Next, as shown in FIG. 13, movement of carriages 120, 130 and 140 is unlocked using lever 114 and locking arms 131 and 141. Such unlocking allows translational movement of all carriages and rotational movement of carriage 140. While casters 112a-112d remain locked, carriage 140 is moved transversely to move projections 145a and 145b into openings defined by sled 420 and rail 422a. Carriages 120 and 130 may also be moved, and carriage 140 tilted, to align projections 145a and 145b with the openings. To facilitate this alignment, some embodiments exhibit an amount of play in the rotational degrees of freedom and free-floating of the translational degrees of freedom. Relative balancing of the available degrees of freedom reduces a need for the operator to overcome counterforces within the system during positioning.

Once in position, projections 145a and 145b are rotated to clamp carriage 140 to sled 420. Any type of mechanical interface to fixedly couple carriage 140 to table 400 may be used in some embodiments, including non-clamping interfaces. From the clamping point, three translational degrees of freedom and a pitch degree of freedom adjacent to rail 422a are available. In some embodiments, rail 422a is sufficiently long and stiff to cause the pitch degree of freedom to track the pitch of table 400. The clamping interface need not exhibit zero backlash provided that the span between projections 145a and 145b is long enough to avoid excess perturbation due to backlash or due to the initial loads on the interface always occurring in one direction.

After the coupling of carriage 140 to table 400, and due to the unlocking of carriages 120, 130 and 140, arm 200 and drive 300 will move in correspondence with subsequent motions of table 400. Moreover, arm 200 may be controlled to move drive 300 to a suitable position with respect to a patient disposed on table 400 without disturbing the position of table 400 or of any element of cart 100.

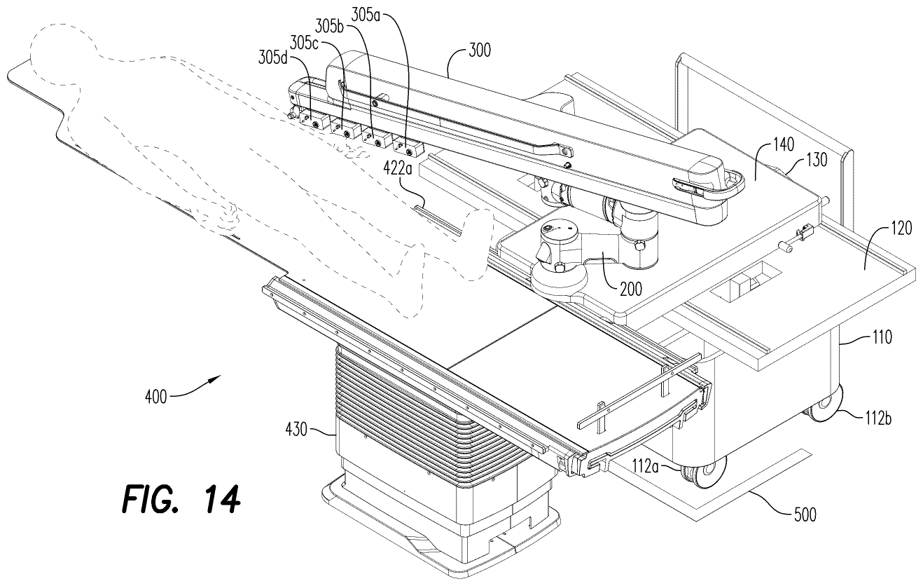

FIG. 14 is a perspective view of cart 100 coupled to table 400 in an initial position in accordance with some embodiments. The initial position simply reflects a vertical, longitudinal, transverse and rotational position of support 410 at a time of initial coupling of carriage 140 to table 400. This initial position also corresponds to a given vertical position of carriage 120, a longitudinal position of carriage 130, and transverse and rotational positions of carriage 140. According to some embodiments, a patient is disposed on table 400 prior to coupling carriage 140 to table 400,

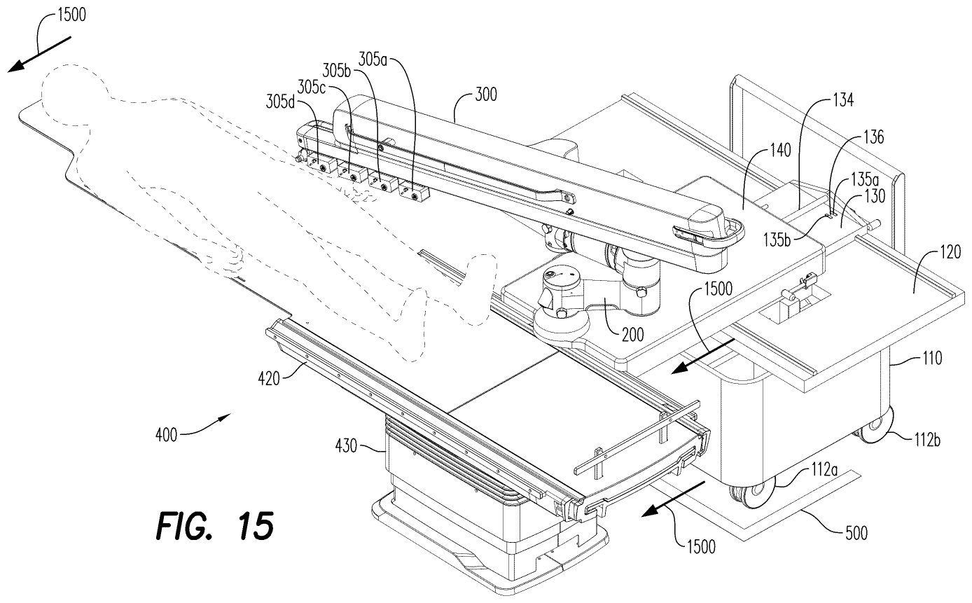

FIG. 15 is a perspective view of cart 100 coupled to table 400 after transverse movement of support 410 from the initial position shown in FIG. 14. Specifically, pedestal 430 is operated to move sled 420 and support 410 in the direction of arrow 1500. Due to the coupling of carriage 140 to table 400, carriage 140 also moves in the direction of arrow 1500. As a result, the positions of cassettes 305a-305d relative to support 410 (and to an otherwise-stationary patient located on support 410) remains unchanged.

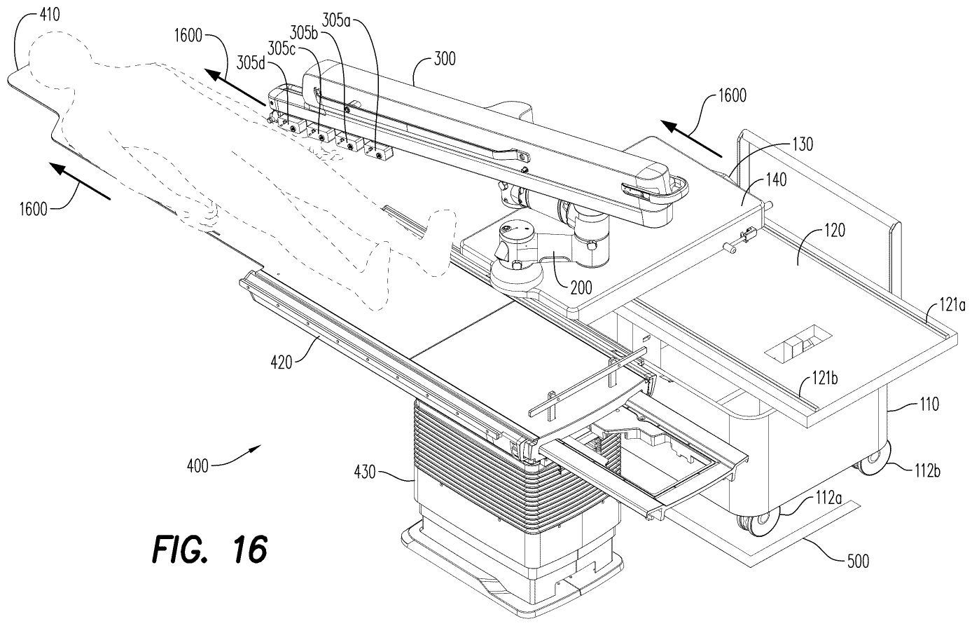

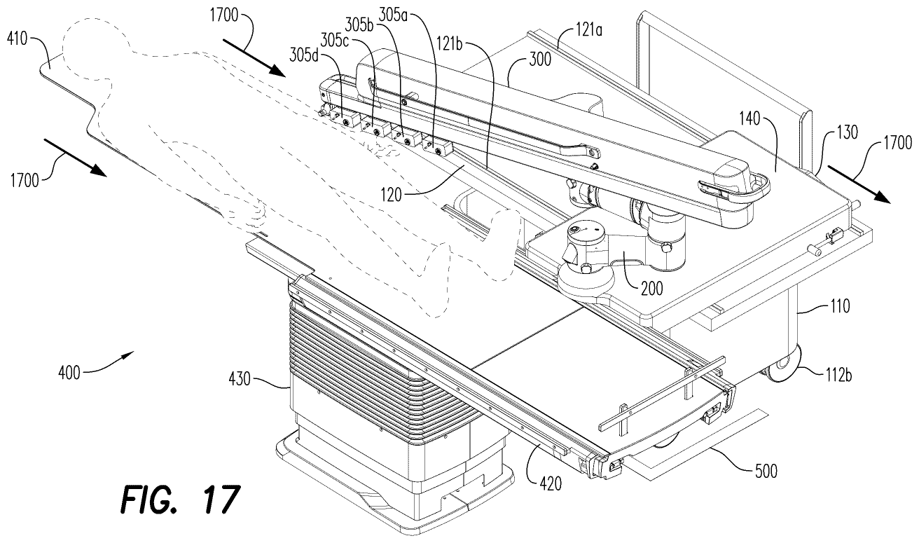

FIG. 16 is a perspective view of cart 100 after longitudinal movement of support 410 in the direction of arrow 1600.from the initial position shown in FIG. 14. Since carriage 140 is coupled to table 400 during this movement, the movement causes carriage 130 to move longitudinally along carriage 120 and to carry carriage 140, arm 200 and drive 300 in the longitudinal direction. The position of cassettes 305a-305d relative to support 410 again remains unchanged despite of the longitudinal movement. FIG. 17 illustrates movement of support 410 in an opposite longitudinal direction depicted by arrow 1700. This movement causes carriage 130, carriage 140, arm 200 and drive 300 to also move longitudinally with respect to carriage 120.

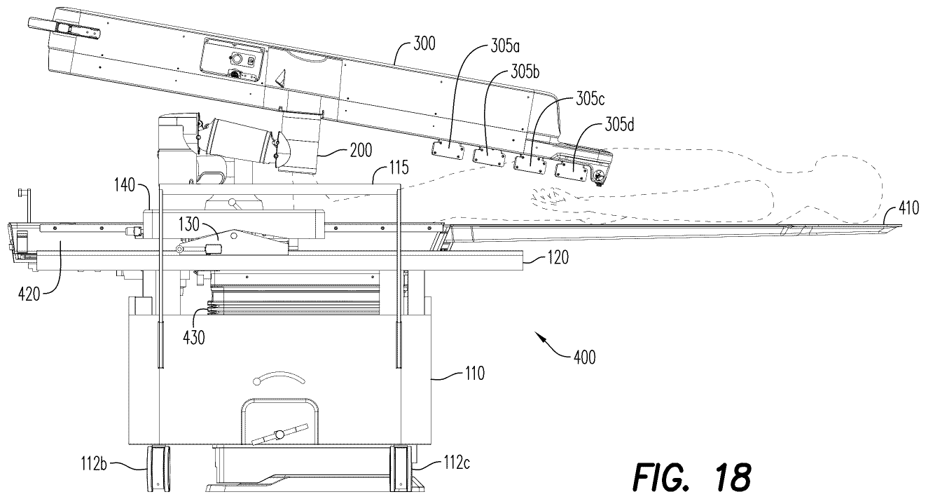

FIG. 18 is an elevation view of table 400, cart 100, arm 200 and drive 300 in accordance with some embodiments. Cart 100 has been coupled to table 400 and arm 200 has been manipulated to position cassettes 305a-305d of drive 300 in a desired position with respect to an access site of a patient. The access site may be an artery of the left wrist of the patient. According to some embodiments, robotic drive 300 may then be operated to begin providing a catheter-based interventional procedure to the patient.

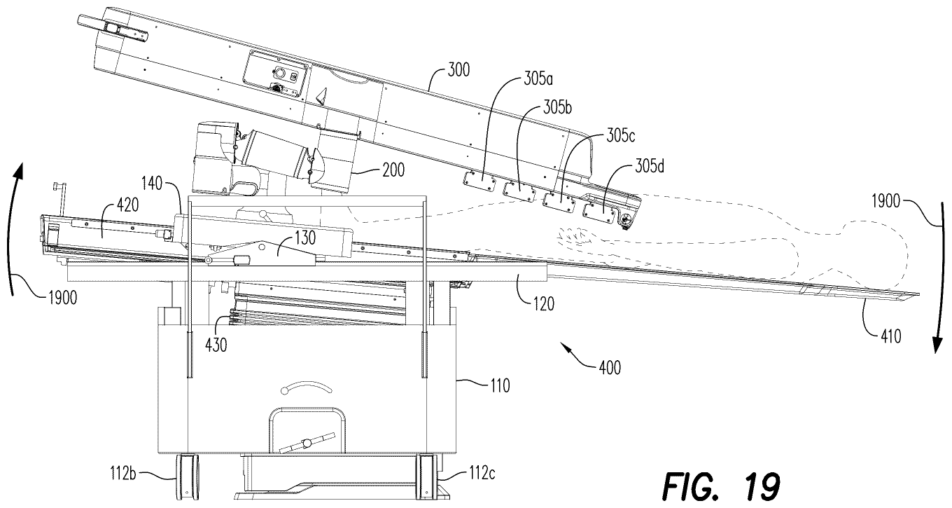

FIG. 19 illustrates rotation, or tilting, of support 410 and sled 420 in the direction of arrow 1900. Pedestal 430 of table 400 may be operated to tilt sled 420 and, as a result, support 410, in the manner shown. The tilting of support 410 and sled 420 causes carriage 140 to rotate about a longitudinal axis of carriage 130, which causes similar rotation of arm 200 and drive 300 about the longitudinal axis. Consequently, cassettes 305a-305d of drive 300 are maintained in the desired position with respect to the access site of the patient.

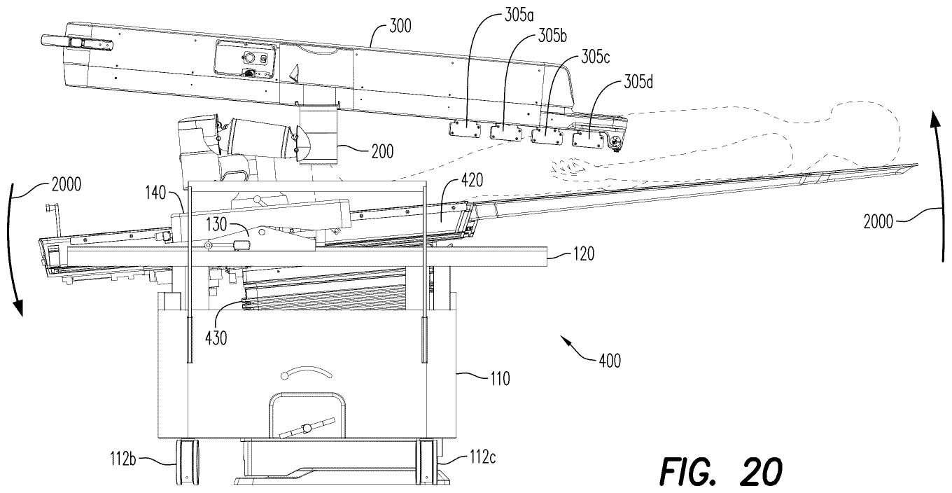

FIG. 20 illustrates rotation of support 410 and sled 420 in the direction of arrow 2000, opposite from the rotation depicted in FIG. 19. Pedestal 430 of table 400 has been operated to rotate sled 420 and support 410, which causes carriage 140 to rotate about the longitudinal axis of carriage 130. Again, the responsive rotation of carriage 140 results in maintaining the position of cassettes 305a-305d relative to the access site of the patient.

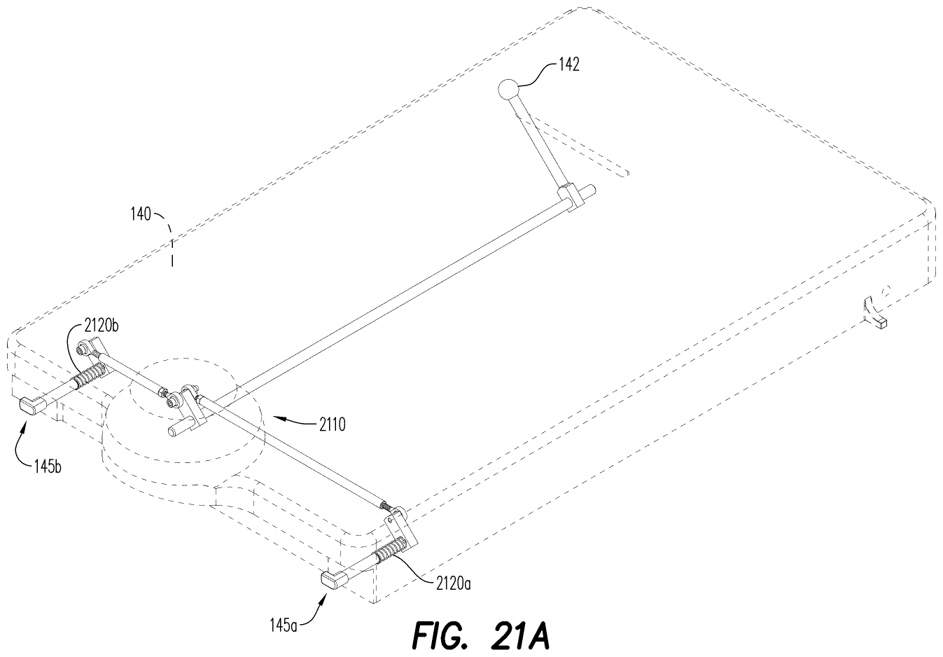

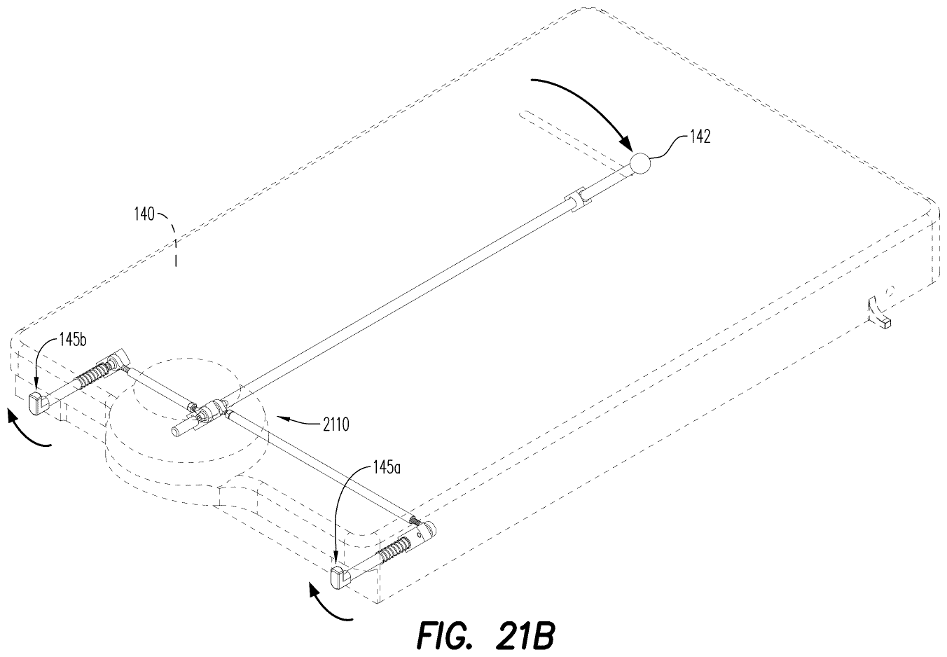

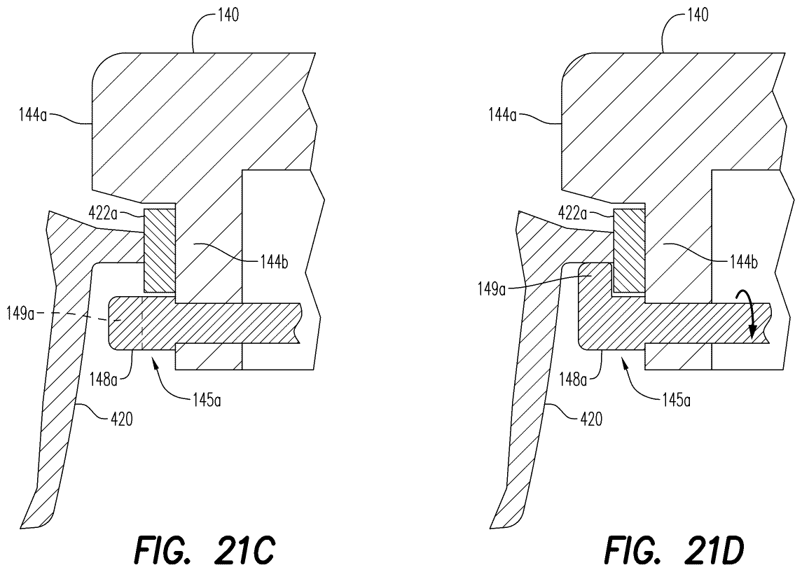

FIGS. 21A and 21B depict a mechanism for coupling a carriage to a table in accordance with some embodiments. FIG. 21A shows actuator 142 in an unactuated position corresponding to a horizontal orientation of projections 145a and 145b. Actuator 142 is coupled to linkage 2110. Actuator 142 and linkage 2110 are biased toward the state depicted in FIG. 21A by virtue of springs 2120a and 2120b, but embodiments are not limited thereto FIG. 21B shows actuator 142 and linkage 2110 after movement (e.g., manually by an operator) of actuator 142 to an actuated position. The movement causes linkage 2110 to rotate projections 145a and 145b to the depicted vertical positions.

FIG. 21C is a cross-sectional view of an interface between sled 420, rail 422a and carriage 140 prior to coupling cart 100 to table 400 according to some embodiments. Projection 145a is disposed in a horizontal position, and it is assumed that actuator 142 is positioned as shown in FIG. 21A. Lower ledge 144b of carriage 140 abuts rail 422a and upper ledge 144a hangs over rail 422a and a portion of sled 420. Actuator 142 is then moved to the actuated position of FIG. 21B, causing projection 145a to rotate into the position shown in FIG. 21D.

In FIG. 21D, portion 149a of projection 145a is adjacent to rail 422a and to a portion of sled 420 connected to rail 422a. Portion 148a is also adjacent to rail 422a. Generally, a clamping structure has been formed about rail 422a consisting of an underside of upper ledge 144a, lower ledge 144b, portion 149a and portion 148a. As a result, any vertical, longitudinal, transverse or tilting movement of rail 422a is imparted to carriage 140.

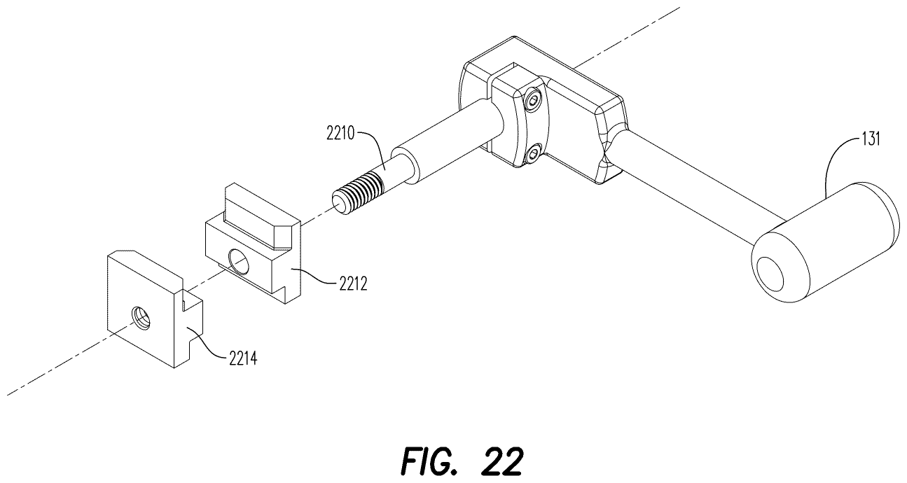

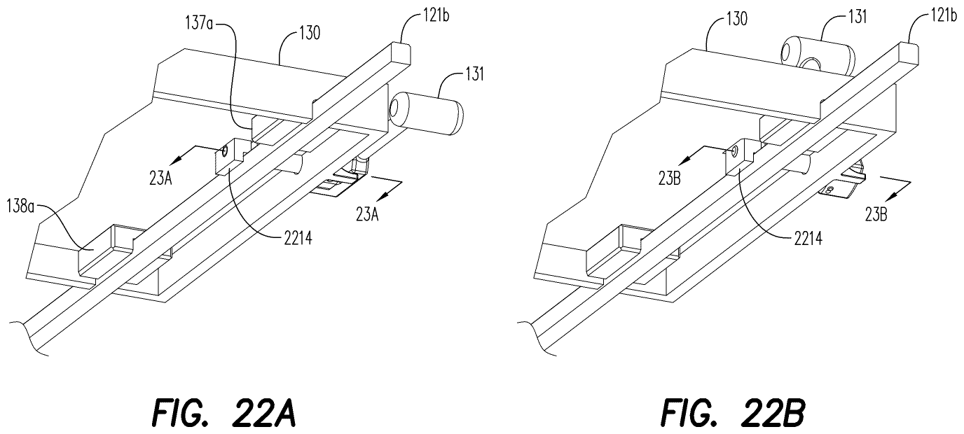

FIG. 22 is a perspective view of locking arm 131 in accordance with some embodiments. Locking arm 131 includes threaded extension 2210 which passes through clamping plate 2212 and mates with threads of clamping plate 2214.

FIGS. 22A and 22B illustrate mechanisms for facilitating and preventing longitudinal movement of carriage 130 in accordance with some embodiments. Bearings 2222a and 2224a are coupled to an underside of carriage 130. Channels within bearings 2222a and 2224a accept rail 121b and allow carriage 130 to move smoothly along rail 121b. A similar bearing arrangement may exist for moving carriage 130 along rail 121a (not shown in FIG. 22A).

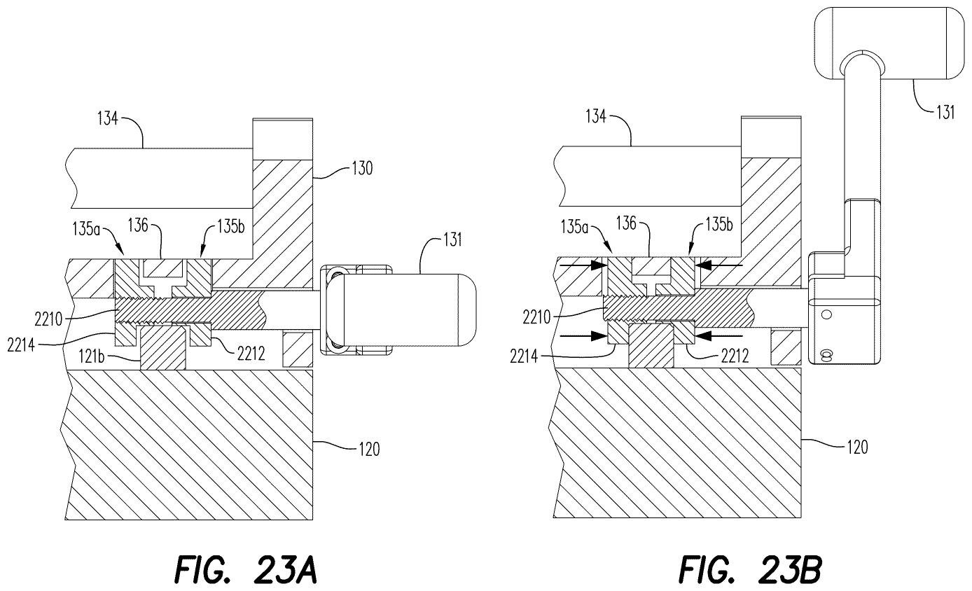

Locking arm 131 is in an unlocked position in FIGS. 22A and 23A. In the unlocked position, clamping plates 2212 and 2214 do not contact rail 121b. Upper portions of clamping plates 2212 and 2214 reside in openings 135a and 135b of carriage 130, on either side of carriage portion 136. During movement of arm 131 from the unlocked position of FIGS. 22A and 23A to the locked position of FIGS. 22B and 23B, threaded extension 2210 advances through clamping plate 2214 to move clamping plates 2212 and 2214 closer to one another until clamping plates 2212 and 2214 firmly engage rail 121b and carriage portion 136. This engagement resists movement of carriage 130 from its current position along rail 121b.

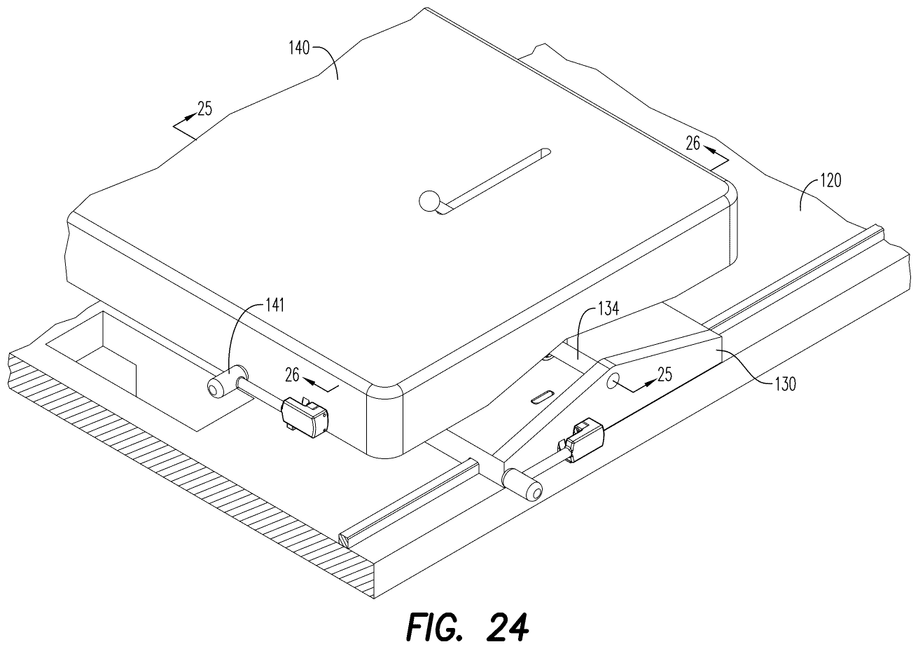

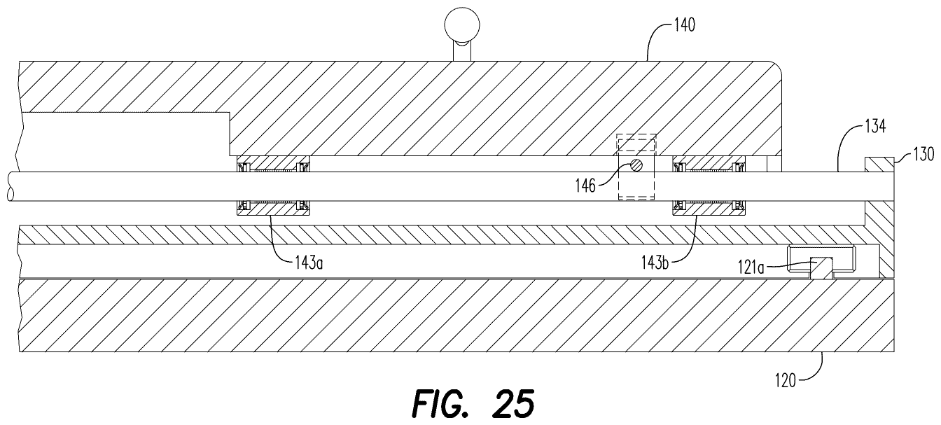

FIGS. 24 and 25 illustrate mechanisms providing rotation and transverse movement of carriage 140 in accordance with some embodiments. Generally, with locking arm 141 in the unlocked position shown in FIG. 24, carriage 140 may rotate about and move along a long axis of support bar 134. The long axis of support bar 134 is substantially perpendicular to patient table 400 when cart 100 is coupled thereto.

Linear bearings 143a and 143b are coupled to an underside of carriage 140. Linear bearings 143a and 143b are coupled to support bar 134 in a manner which allows carriage 140 to rotate about bar 134 and to move along the long axis of bar 134. Additional unshown linear bearings may be similarly coupled to the underside of carriage 140 and to support bar 134.

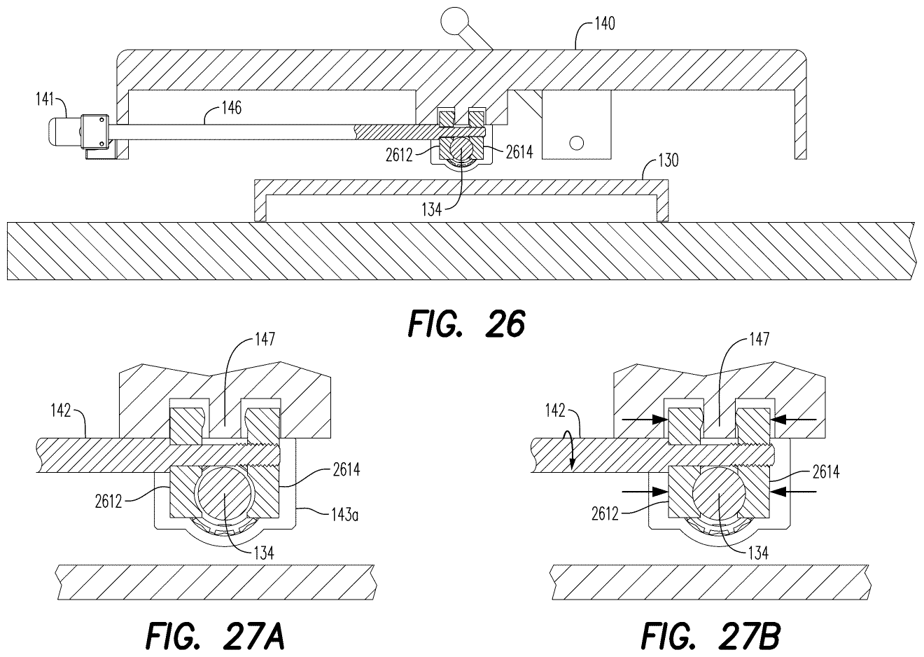

FIGS. 26, 27A and 27B illustrate locking the movement of a carriage in accordance with some embodiments. FIG. 26 shows locking arm 141 in an unlocked position and actuation bar 146 coupled thereto. As further shown in FIG. 26 and in detail in FIG. 27A, actuation bar 146 includes a threaded extension which may be constructed similarly to threaded extension 2210. The threaded extension of actuation bar 146 passes through an opening of clamping plate 2612 and is threaded into female threads of clamping plate 2614. While locking arm 141 is in the unlocked position, clamping plates 2612 and 2614 are separated from bar 134 and from portion 147 of carriage 140, allowing carriage 140 to move freely along bar 134 as provided by bearings 143a and 143b.

As illustrated in FIG. 27B, movement of locking arm 141 into the locked position rotates actuation bar 146. This rotation causes the threaded extension of bar 146 to advance through clamping plate 2614 and to thereby bias clamping plates 2612 and 2614 in the directions of the arrows. The biasing causes clamping plates 2612 and 2614 to firmly engage both bar 134 and carriage portion 147, resisting translational and rotational movement of carriage 140 with respect to bar 134.

While only certain features of some embodiments have been illustrated and described herein, many modifications and changes will occur to those in the art. It is, therefore, to be understood that the appended claims are intended to cover all such modifications and changes. The features described herein may be combined in multiple combinations such that a feature may be used alone or in any combination with any of the other features.

What is claimed is:

1. A system for supporting a robotic drive, the system comprising:
a base,
a first carriage coupled to the base and configured to move in a first direction with respect to the base;
a second carriage coupled to the first carriage and configured to move along the first carriage in a second direction substantially perpendicular to the first direction;
a third carriage coupled to the second carriage and configured to move along the second carriage in a third direction substantially perpendicular to the first direction and to the second direction; and
a tilting mechanism comprising a table interface,
wherein movement of a table coupled to the table interface in the first direction causes the first carriage to move in the first direction,
wherein movement of the table coupled to the table interface in the second direction causes the second carriage to move in the second direction,
wherein movement of the table coupled to the table interface in the third direction causes the third carriage to move in the third direction, and
wherein tilting of the table causes the tilting mechanism and the robotic drive to tilt.

2. A system according to claim 1, wherein the first direction is vertical with respect to a surface of the table, the second direction is longitudinal with respect to the surface of the table, and the third direction is transverse with respect to the surface of the table.

3. A system according to claim 1, wherein the third carriage comprises the tilting mechanism and the robotic drive is mounted on the third carriage, and
wherein tilting of the table causes the third carriage and the robotic drive to tilt with respect to the second carriage.

4. A system according to claim 3, wherein the tilting of the table and of the third carriage are to a same degree.

5. A system according to claim 3, the third carriage comprising an actuator to actuate the table interface to couple the third carriage to the table.

6. A system according to claim 5, the table interface comprising one or more projections actuatable by the actuator.

7. A system according to claim 1, the first carriage comprising at least one rail on which the second carriage moves, and the second carriage comprising a first locking arm actuatable to lock the second carriage to the at least one rail.

8. A system according to claim 7, the second carriage comprising a bar disposed in the third direction and on which the third carriage rotates, and the third carriage comprising a second locking arm actuatable to prevent rotation of the third carriage about the bar.

9. A system according to claim 8, the second locking arm actuatable to prevent movement of the third carriage along the second carriage in the third direction.

10. A system according to claim 9, the second locking arm actuatable to lock the second carriage to the bar.

11. A system for supporting a robotic drive, the system comprising:
a base,
a first carriage coupled to the base, configured to move vertically with respect to the base and comprising a first mechanism actuatable to prevent vertical movement of the first carriage;
a second carriage coupled to the first carriage, configured to move longitudinally along the first carriage and comprising a second mechanism actuatable to prevent longitudinal movement of the second carriage; and
a third carriage coupled to the second carriage, configured to move transversely along the second carriage and to rotate about an axis of the second carriage, and comprising a third mechanism actuatable to prevent transverse movement and rotation of the third carriage.

12. A system according to claim 11, the third carriage comprising a table interface and an actuator to actuate the table interface to couple the third carriage to a table.

13. A system according to claim 12, the table interface comprising one or more projections actuatable by the actuator.

14. A system according to claim 12, wherein movement of the table vertically while coupled to the third carriage causes the first carriage to move vertically,
wherein movement of the table longitudinally while coupled to the third carriage causes the second carriage to move longitudinally,
wherein movement of the table transversely while coupled to the third carriage causes the third carriage to move transversely, and
wherein rotation of the table while coupled to the third carriage causes the third carriage to rotate.

15. A system according to claim 14, wherein the rotation of the table and of the third carriage are to a same degree.

16. A system according to claim 11, the first carriage comprising at least one rail on which the second carriage moves, and the first mechanism actuatable to secure the second carriage to the at least one rail.

17. A system according to claim 16, the second carriage comprising a bar disposed in the third direction and on which the third carriage rotates, and the second mechanism actuatable to prevent rotation of the third carriage about the bar.

18. A system according to claim 17, the second mechanism actuatable to secure the third carriage to the bar.

19. A method for coupling a system comprising a base, a first carriage, a second carriage and a third carriage to a table, comprising:
moving the base adjacent to a table;
after moving the base adjacent to the table, moving the first carriage vertically with respect to the base;
after moving the first carriage vertically with respect to the base, moving the second carriage longitudinally with respect to the base;
after moving the second carriage longitudinally with respect to the base, moving the third carriage transversely with respect to the base to place a table interface of the third carriage adjacent to the table; and
actuating the table interface to couple the third carriage to the table.

20. A method according to claim 19, further comprising:
before moving the first carriage vertically with respect to the base, actuating a first mechanism to allow vertical movement of the first carriage with respect to the base;
before moving the second carriage longitudinally with respect to the base, actuating a second mechanism of the second carriage to allow longitudinal movement of the first carriage with respect to the base;
before moving the third carriage transversely with respect to the base, actuating a third mechanism of the third carriage to allow transverse movement of the third carriage with respect to the base.

21. A method according to claim 20, wherein actuating the third mechanism of the third carriage allows rotation of the third carriage with respect to the second carriage.

22. A method according to claim 20, wherein moving the base adjacent to the table comprises aligning the table interface with a rail of the table.
